(19) United States
(12) Patent Application Publication
(10) Pub. No.: US 2002/0156587 A1
(43) Pub. Date: Oct. 24, 2002

(54) Method of analyzing gene expression data using fuzzy logic

(76) Inventors: Peter James Woolf, Ann Arbor, MI (US); Yixin Wang, San Diego, CA (US)

Correspondence Address: PILLSBURY WINTHROP, LLP, P.O. BOX 10500, MCLEAN, VA, 22102, US

(21) Appl. No.: 09/778,956

(22) Filed: Feb. 8, 2001

Related U.S. Application Data
(60) Provisional application No. 60/181,477, filed on Feb. 10, 2000.

(51) Int. Cl. G06F 19/00; G01N 33/48; G01N 33/50

(52) U.S. Cl. 702/20; 435/440

(57) ABSTRACT

An embodiment of the invention provides a method and system for managing and analyzing information obtained from differential expression of genetic information in biological cells. Crisp input data are received from sets of expression data from control and treatment cell-derived samples representing a direction and a magnitude of regulation of each one of a higher number of different genes or proteins. The crisp input data are fuzzified to provide fuzzified values. A set of heuristic rules is applied to the fuzzified values to generate a predicted value of a data point C. The predicted value of the data point C is defuzzified. Finally, a confidence level of the predicted value of C is determined.

### RESERVATION OF COPYRIGHT

This patent document contains material subject to copyright protection. The copyright owner has no objection to the facsimile reproduction by anyone of the patent document, as it appears in the U.S. Patent and Trademark Office patent files or records, but otherwise reserves all copyright rights whatsoever.

### BACKGROUND OF THE INVENTION

1. Field of the Invention

The invention relates to a data processing system and method of use for analyzing gene expression data using a fuzzy logic-based computer algorithm.

2. Description of Background Information

Cells regulate the expression of their genes in response to environmental changes. Normally this regulation is beneficial to the cell, protecting it from starvation or injury; however errors in this regulation can lead to serious diseases ranging from cancer to heart disease. Measuring the differential expression of genes from various stages of an organism's development, different tissues, and organisms subjected to different stresses provides information instrumental in understanding the relationships between genes and their functions. Gene regulation is useful for both assaying drugs and as a source of new molecular targets, assuming the regulatory network controlling a given gene is well understood. As such, changes in gene expression patterns can be used to assay drug efficacy throughout the drug discovery process.

One assay that takes advantage of the existing level of sequence information, and that is complementary to sequence and genetic analysis, is gene expression profiling. Expression profiling can be carried out by one of a number of different technologies, such as commercially or privately manufactured gene chips, which typically measure the expression level of thousands of genes simultaneously using an array of oligonucleotides bound to a silicon surface. These arrays are hybridized under stringent conditions with a complex sample representing mRNAs expressed in the test cell or tissue. Target sequences hybridize to immobilized oligonucleotides and are typically detected via fluroescent labels. Relative intensity levels of fluorescent labels indicate relative gene expression in a given sample obtained from a source subjected to a particular condition. As a sample source is subjected to a variety of conditions, a given gene will display a profile under these conditions. Thus, gene profiles represent a vector of gene expression intensities corresponding to multi-conditional gene expression patterns. The results from these expression profiling technologies are quantitative and highly parallel, thereby allowing an accurate snapshot to be made of the workings of the cell in a particular state.

Since thousands of hybridization reactions may occur in a single array, expression profiling assays generate huge data sets that are not amenable to simple analysis. To maximize the use of such data, efforts are underway to develop algorithms interpreting and interconnecting results for different genes under different conditions. Currently, expression data is typically analyzed using clustering techniques comprising algorithms that identify distinct expression patterns by grouping genes with similar expression patterns (Tavazoie,1999; Cho, 1998). An example of such a clustering algorithm is CAST (Cluster Affinity Search Technique), which uses a graph theoretic approach and employs a stochastic model of input (Yakhini, 1998). Cluster analysis of multi-conditional gene expression patterns generally involve the steps of i) measuring gene expression levels reported as a vector of real numbers; ii) computing a similarity matrix for the measured genes; iii) clustering genes based on their similarity to each other; iv) visually representing the clusters; and v) analyzing the results obtained therefrom.

Clustering, however, can only distinguish between those genes that have the same and different expression profiles. Genes in any given cell make up a complex network that cannot be revealed with current techniques such as clustering, etc. In order to determine networks describing how various genes interrelate, more elaborate data mining techniques are needed.

Fuzzy logic uses techniques drawn from engineering and other applied sciences for controlling systems as diverse as washing machines to auto-focus cameras (Cox, 1992; Zadeh, 1974). The concept of fuzzy logic is based on the “fuzzy estimation” of human thinking, as opposed to precise mathematical computation. Fuzzy logic provides a means for transforming precise numbers, such as 12.433, into qualitative descriptors, such as HIGH in a process called “fuzzification”. Thus, discrete input values may be converted into a range of values. Once transformed, such qualitative data may be analyzed using heuristic rules, which in turn generate fuzzy solutions. For example, the heuristic rule “if HOT then move FAST” takes HOT as a fuzzy input and FAST as a fuzzy solution. In another process called “defuzzification”, this heuristic solution can be transformed from a qualitative descriptor back into a precise number.

3. Definitions

For purposes of clarification, and to facilitate an understanding of the present invention and the embodiments disclosed herein, a number of terms used herein are defined as follows:

Expression Profiling

A process by which molecular techniques are used to measure and compare expression levels of certain nucleic acid sequences (e.g., mRNAs, genes, expressed sequence tags (ESTs)), or levels of certain gene products, such as amino acid sequences (protein or fragments of proteins), in a cell-derived sample in relation to the levels of the same nucleic acid sequences or gene products from a different sample, or from the same sample measured at a different time point.

Gene

A sequence of nucleotides specifying a particular polypeptide chain.

Gene Product

One or several polypeptide chains of amino acids translated from RNA transcribed from a gene.

Activator/Repressor

Proteins, such as transcription factors and tumor suppressors, capable of inducing physiological change, usually enhancement or inhibition of gene expression in a given biological system (activation or deactivation), typically in response to an environmental stress.

mRNA

Messenger Ribonucleic acid.

### SUMMARY OF THE INVENTION

An embodiment of the invention provides a method for managing and analyzing information obtained from differential expression of genetic information in biological cells. Crisp input data is received from sets of expression data from control and treatment cell-derived samples representing a direction and a magnitude of regulation of each one of a higher number of different genes or proteins. The crisp input data is fuzzified to provide fuzzified values. A set of heuristic rules is applied to the fuzzified values to generate a predicted value of a data point C. The predicted value of the data point C is defuzzified. Finally, a confidence level of the predicted value of C is determined.

A second embodiment of the invention provides a system for managing and analyzing information obtained from differential expression of genetic information in biological cells. The system includes a data receiver for receiving crisp input data from control and treatment cell of cell-derived samples. A fuzzifier fuzzifies the crisp input data to provide fuzzified values. A heuristic rules applier applies a set of heuristic rules to the fuzzified values to generate a predicted value of a data point C. A defuzzifier defuzzifies the predicted value of C. Finally, a confidence level determiner determines a confidence level of the predicted value of C.

A third embodiment of the invention provides a machine-readable medium having recorded thereon machine-readable information. When the machine-readable information is loaded into a computer memory and executed by a computer, the machine-readable information causes the computer to: receive crisp input data from sets of expression data from control and treatment sets of cell-derived samples representing a direction and a magnitude of regulation of each one of a higher number of different genes or proteins; fuzzify the crisp input data to provide fuzzified values; apply a set of heuristic rules to the fuzzified values to generate a predicted value of a data point C; defuzzify the predicted value of C; and determine a confidence level of the predicted value of C.

### BRIEF DESCRIPTION OF THE DRAWINGS

Illustrative embodiments of the invention are further illustrated in the accompanying drawings in which like reference numerals represent the same or similar elements throughout the several views of the drawings.

FIG. 1 depicts fuzzy membership as a function of a normalized expression level.

FIG. 2 shows a decision matrix describing an activator (A) and repressor (B) acting on a target (C).

FIGS. 3A-3C show the fuzzy logic prediction for CYB2 (A), HAP1 (B) and CYC1 (C).

FIG. 4 shows the HAP 1 and ROX1 regulatory network as predicted by the yeast expression data set.

FIG. 5 is a block diagram of an expression profiling data analysis system.

FIG. 6 is a block diagram which illustrates the analyzer in detail.

FIG. 7 provides a more detailed view of the confidence level determiner of FIG. 6.

FIG. 8 provides a more detailed view of the filter of FIG. 6.

FIG. 9 provides a more detailed view of the scorer of FIG. 6.

FIG. 10 is a flow diagram of a gene expression profiling process.

FIG. 11 is a flow diagram of an embodiment of the analyzer system.

### DETAILED DESCRIPTION

Three main advantages favor the application of fuzzy logic to the analysis of gene expression data over previously known techniques. First, fuzzy logic inherently accounts for noise in data because the algorithm extracts trends, not precise values. Second, in contrast to other automated decision making algorithms, such as neural networks or polynomial fits, algorithms in fuzzy logic are cast in the same language used in day-to-day conversation. As a result, predictions made using fuzzy logic are easily interpreted and can be extrapolated in predictable ways. Third, fuzzy logic techniques are computationally efficient and can be scaled to include an unlimited number of components. Thus, they are able to recognize a large number of biologically important patterns.

One embodiment of the invention comprises a data processing system, comprising an analysis system 10, as shown in FIG. 5. An analyzer 27, included within host computer 24, is used for carrying out certain analysis processes associated with expression profiling and managing data acquired from the expression profiling. Analyzer 27 comprises fuzzy logic that can identify logical relationships between genes, and in some cases, even predict the function of an unknown gene.

This algorithm was validated using yeast expression data gathered from the Affymetrix GeneChip® system. By using yeast gene expression data collected at different time points of the cell cycle, the illustrated analyzer 27 permits identification of many regulatory elements and their target genes within the yeast cell that work together to maintain and control the “state” of the cell. Several cases were validated by available experimental results, including the signaling network controlled by the transcription factors HAP1 and ROX1, known to control the transition in yeast from anaerobic to aerobic growth. These results suggest that the fuzzy logic technique of the illustrated embodiment can effectively find biologically relevant connections between sets of genes, which in turn aids in describing the complex web of interactions that regulate gene expression.

Referring now to the drawings in greater detail, FIG. 5 shows an analysis system 10 according to the illustrated embodiment of the present invention. An expression profiling subsystem 12 is provided, which is coupled to a client computer 14. In the illustrated embodiment the expression profiling subsystem 12 is coupled via intranet 22; however, other methods, such as, but not limited to, direct coupling may also be employed. Client computer 14 comprises, among other elements, a browser application 16, a human interface 18, and a display 20. Human interface 18 may comprise any standard or other interface for facilitating human interaction with and control of client computer 14, including, for example, a keyboard and a mouse. Client computer 14 is coupled to a host computer 24 via, for example, a network connection illustrated in FIG. 5 as the intranet 22. Host computer 24 is connected to a database 26.

Expression profiling system 12 may comprise, for example, an Affymetrix cDNA array. It generates, from control and treatment sets of cell-derived samples, respective sets of sequence data representing a direction and a magnitude of regulation of each one of a high number of different nucleic acid sequences.

Client computer 14, together with human interface 18, display 20, and browser application 16, allow a user to operate analysis system 10. Client computer 14 Expression profiling subsystem 12 obtains the expression profiling data and stores that data in an organized fashion on database 26. Host computer 24 is provided with, among other elements, the analyzer 27 for carrying out certain analysis process steps associated with expression profiling and managing the data acquired from the expression profiling. A database server software component 28 is provided for handling and acting on database queries and responses.

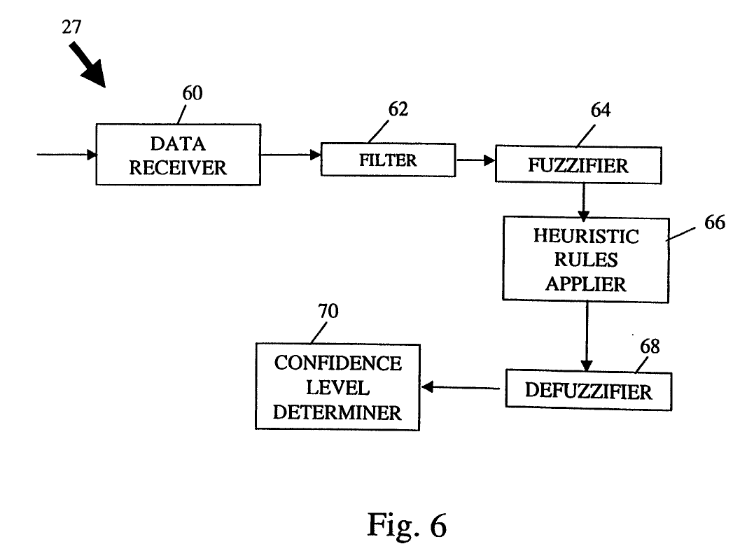

FIG. 6 shows the analyzer 27 in more detail. The analyzer receives crisp input data, from sets of expression data, into data receiver 60. Optionally, the data is filtered by filter 62. The filtered data is then fuzzified, by fuzzifier 64, into fuzzy values. In an embodiment of the invention, the fuzzy values have high, medium and low components. The fuzzy values are applied, by heuristic rules applier 66, to a decision matrix. The heuristic rules applier 66 provides a fuzzy value for a predicted value of a data point C. Defuzzifier 68 defuzzifies the fuzzy value of C. Confidence level determiner 70 determines a level of confidence in the predicted value of C.

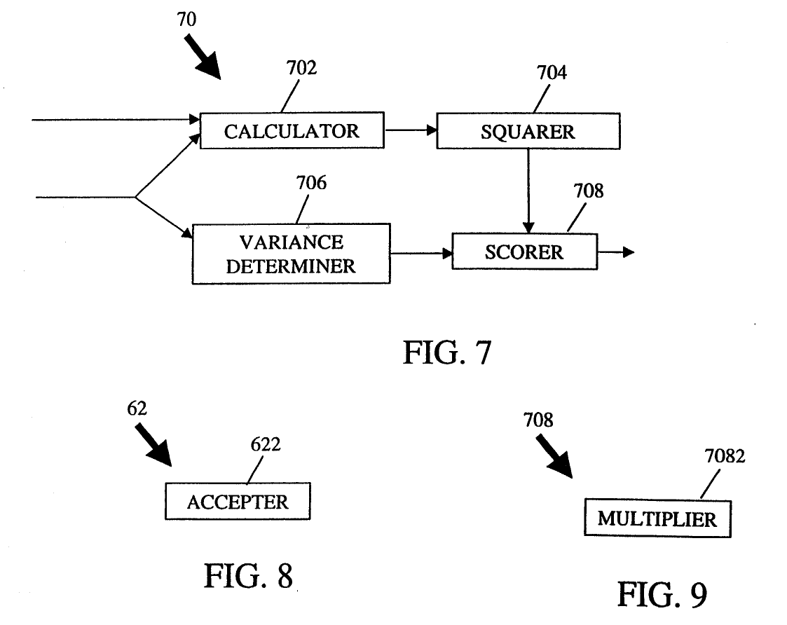

FIG. 7 is a more detailed view of the confidence level determiner 70. The confidence level determiner includes a calculator 702 for calculating a difference r between the defuzzified predicted value of a data point C and an observed value of C. Squarer 704 squares r to provide r². Scorer 708 includes a multiplier 7082 (see FIG. 9) for multiplying a distribution variance by r² to provide a general score for predicting credibility of the predicted value of C.

FIG. 8 shows a more detailed view of filter 62. The filter 62 includes an accepter 622. The accepter 622 accepts crisp input data only when one of the crisp input data, having a value greater than all other ones of the crisp input data, is at least three times larger than another one of the crisp input data having a value less than all other ones of the crisp input data.

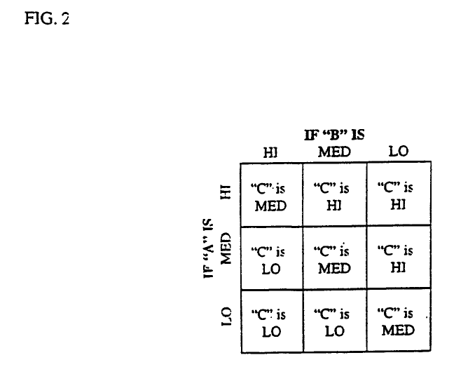

The heuristic rules applier 66 applies heuristic rules, as illustrated by FIG. 2, which shows the heuristic rules as applied to a decision matrix. The heuristic rules of FIG. 2 illustrate, for example, an activator/repressor model, although other models may also be used depending upon the type of relationship under study. Assuming A is an activator, B is a repressor and C is a target, the matrix of FIG. 2 shows the expected relationships. For example, when A is HI and B is HI, C is MED. When A is HI and B is MED, C is HI. When A is HI and C is LO, C is HI. When A is MED and B is HI, C is LO. When A is MED and B is MED, C is MED. When A is MED and B is LO, C is HI. When A is LO and B is HI, C is LO. When A is LO and B is MED, C is LO. Finally, when A is LO and B is LO, C is MED.

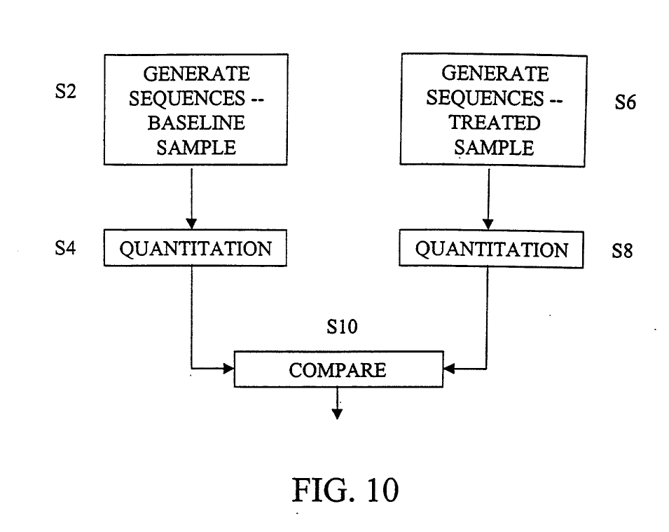

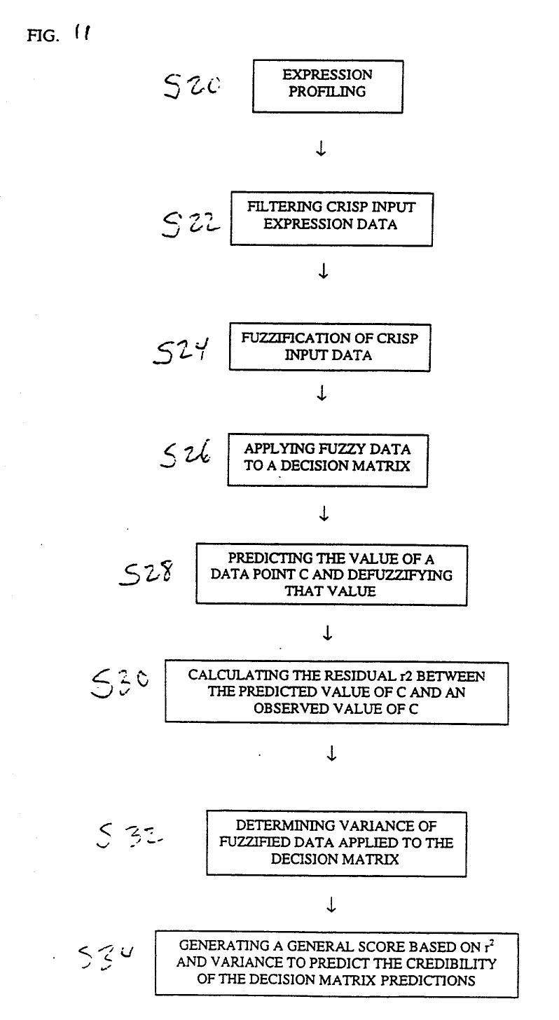

FIG. 10 generally shows an expression profiling process in accordance with the illustrated embodiment. In an initial act S2, gene expression values are generated based upon a baseline sample (otherwise referred to as a control sample) of cells. In an act S6, one or more gene expression values may be generated based upon treated samples, i.e., samples of cells based upon those cells entering into a diseased state or being treated with a particular compound. After performing each of acts S2 and S6, quantitation is performed.

In order to determine whether gene expression levels have been substantially affected (i.e., either repressed or induced), the expression level of the genes generated in a baseline sample is compared with the expression level of the genes generated and grouped in the treated sample or samples, to produce, for each treated sample, an indication of whether a gene was regulated and the extent and direction of that regulation.

More specifically, by way of example, a sample of RNA may be monitored using an expression profiling array, such as an Affymetrix GeneChip™ probe array having, for example, oligonucleotides corresponding to the human genome, capable of detecting expression levels for over 6,000 sequences for that genome. Affymetrix provides a GeneChip™ fluidics station that automates the hybridization of nucleic acid targets to a probe array cartridge, and thus controls the delivery of reagents and the timing and temperature for hybridization. Each fluidics station can independently process four probe arrays at a given time.

Accordingly, each target may be prepared from a set of cell dishes by isolation of RNA over a course of time. The treatment of those cells may be emulated by adding, for example, serum thereto. At predetermined intervals, a small amount of the fluid is removed, and the cells are put in a quiescent state to stop the reaction time. Accordingly, a large set of targets, having a predetermined amount of liquid (e.g., .5 ml each) is produced. The GeneChip™ fluidics station will then automatically hybridize each target, i.e., it will extract all the RNA and label the RNA by adding a chemical tag to each molecule, and control the delivery of the resulting liquid to the probe arrays to facilitate obtaining sequencing information regarding the mRNAs. This is done by the probe arrays exposing the target to light at a predetermined location and measuring the photons collected at various locations within the arrays. The amount of mRNA is then ascertained based upon the signal strength of the reading given by the probe at the appropriate location corresponding to that sequence or sequence segment. A net change in signal may indicate activation or repression of gene transcription, or possibly post-transcriptional stabilization the mRNA due a given set of treatment conditions.

The expression data so obtained are then filtered to create suitable sets of data for analysis according to the invention, essentially as diagrammed in FIG. 11. Such filtering serves to ensure selected data are above a minimum noise threshold and represent gene whose net change in expression satisfies a predetermined level of signal strength.

The fuzzy logic algorithm may also be used to analyze data sets generated by translated products of genes expressed in biological cells under control and treatment conditions. For example, the ProteinChip™ System (Ciphergen Biosystems, Palo Alto, Calif.) consists of microchips having activated surfaces that allow immobilization of antibodies, receptors or DNA for capturing proteins from a sample. After sample binding, the array is washed to remove unbound molecules. Thereafter, energy adsorbing molecules are added to facilitate detection captured proteins via, for example, mass spectrometry. Multiple sets of protein expression spectra may be obtained and filtered to create suitable sets of data for analysis according to the illustrated embodiments of the invention.

Certain implementations or embodiments of the invention are further illustrated in the following, nonlimiting example.

### EXAMPLE

Public domain GeneChip® data describing the yeast cell cycle (downloaded from htti://genomics.stanford.edu) was chosen to validate the fuzzy logic algorithm. Since the yeast cell cycle is a tightly regulated process at the genetic level, the expression data was expected to show detectable relationships among different genes that might be detected via a fuzzy logic algorithm. Also, years of experimental work on yeast has generated an extensive body of biological data describing many of the proteins in the organism, allowing the confirmation or dismissal of findings based on data in the literature.

In this illustrated embodiment, at S20, GeneChip data were obtained from a set of 17 experiments in which data points were profiled under various biological conditions. Thus, the relationship between proteins in numerous distinct cell types and under various stress conditions were followed to ensure detected relationships between gene products were not the results of an artifact created under a particular experimental condition. Each individual experimental condition was repeated 2 or 3 times, and the averaged results were included as a single experiment in the set of 17 experiments analyzed. All proteins later evaluated for relationship purposes must have been expressed to some measurable degree under all 17 experimental conditions. Preferably, a minimum of 9 experimental conditions should be included, although this is not absolute. No maximum number of experiments is controlling.

Before expression data were analyzed, the data were filtered, at S22, to ensure that 1) the data are above a threshold noise level that is determined by GeneChip® software and 2) the data set includes genes that significantly differ in their level of expression. For the yeast data set, the noise level was assumed to be 30 in average difference; thus the highest member of a particular data set had to exceed the noise level to allow that set into the calculation. In this embodiment, for a set to be selected, its regulation magnitudes must vary over a certain minimum range, so as to ensure that the observed signal change was statistically significant. In the illustrated embodiment, the maximum value in the set was at least 3 times greater than the minimum value. The threshold difference between maximum and minimum gene expression levels depends upon the sensitivity of the detection system collecting the data, and thus may vary according to the means of detection selected. The fuzzy logic algorithm of this example processed only those genes that met both criteria.

In this example, genes were selected that follow the pattern of a gene product (C) controlled by both an activator (A) and repressor (B), although any pattern can be searched for in a pre-selected pattern. Generally, the activator-repressor model provides that the concentration of the target C will be high when the activator is high and the repressor is low. Conversely, when the repressor concentration is high, and the activator is low, the concentration of the target is low. An unlimited number of models may be employed, and thus the invention is not limited to the interactions within triplet subsets as exemplified.

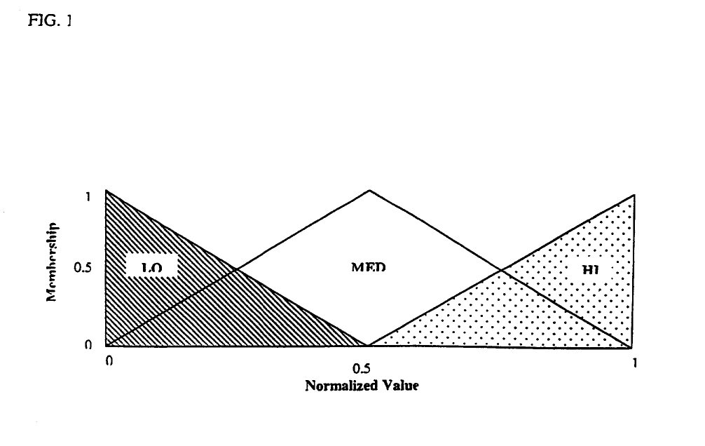

In order to analyze the genetic expression data, the data are transformed, at S24, from crisp values to fuzzy values in a process called “fuzzification.” Data are fuzzified by first normalizing the data from 0 to 1. Thereafter, the normalized value is broken up into various membership classes. For example, FIG. 1 shows the three fuzzy sets used in this algorithm, HI, MED, and LO, as a function of the normalized value. Note that three fuzzy sets are used as an example only and that the number of fuzzy sets need not be three, for example, five, or any other appropriate number may be used. For a normalized value of 0.25, the fuzzy value is 0.5 LO, 0.5 MED, and 0 HI; or put another way, 0.25 is 50% low, 50% medium and 0% high. Accordingly, any crisp, normalized value can be broken down into its membership in each fuzzy class.

After the data are fuzzified, triplets of data are compared, at S26, using a set of heuristic rules in the form of a decision matrix (see FIG. 2). Fuzzified values of A and B are entered into this matrix, and at points where their predictions overlap a score is generated. The goal of this process is to identify values of A and B that overlap and to give that element its appropriate weight. The following pseudocode may be used to apply the fuzzy values to a decision matrix. The matrix is as depicted, wherein “hm” means A is high and B is med.

| | B high | B med | B low |
|---|---|---|---|
| A high | hh | hm | hl |
| A med | mh | mm | ml |
| A low | lh | lm | ll |

Data enter the matrix in the form of fuzzified values, called fuzzA and fuzzB.

/*zero out matrix*/

hh=hm=hl=mh=mm=ml=lh=lm=ll=0;

/*zero out count*/

count=0;

/*look for overlap, and if overlap add to the count*/

/*high A and high B*/

if((fuzzA[HI]*fuzzB[HI]!=0.0)

{

hh=fuzzA[HI]+fuzzB[HI];

count++;

}

/*high A and med B*/

if((fuzzA[HI]*fuzzB[MED]!=0.0)

{

hm=fuzzA[HI]+fuzzB[MED];

count++;

}

/*repeat for all combinations of A and B (total of 9 comparisons)*/

/*normalize scores based on count such that each condition gets equal weight*/

/*Note that the only possible values of count are 4, 3, 2 and 1*/

if(count==4)

{

hh=hh/4;

hm=hm/4;

hl=hl/4:

mh=mh/4;

mm=mm/4;

ml=ml/4:

lh=lh/4;

lm=lm/4;

ll=ll/4:

}

if(count==2)

{

hh=hh/3;

hm=hm/3;

hl=hl/3:

mh=mh/3;

mm=mm/3;

ml=ml/3;

lh=lh/3;

lm=lm/3;

ll=ll/3:

}

if(count==1)

{

hh=hh/2;

hm=hm/2;

hl=hl/2:

mh=mh/2;

mm=mm/2;

ml=ml/2:

lh=lh/2;

lm=lm/2;

ll=ll/2:

}

/*this process can be repeated for all of the experiments to get average values for hh, hm, hl, etc*/

The following pseudocode illustrates S28, predicting the value of a data point C and defuzzifying the value/

/*we need to defuzzify values using, for example, the activator/repressor schema, as shown in the heuristics table of FIG. 2 (or whatever kind of relation we are looking for), which is stored in TempTable[2][2]. TempTable[2][2] is a 3×3 array having elements ranging from TempTable [0][0] through TempTable [2][2]*/

/*If heuristics table shows that a HI result is expected for C, when A is HI and B is HI,

then . . . */

if(TempTable[A_HI][B _HI]==HI) /*schema at A_HI,B_HI is HI*/

{

Cval=Cval+hh; /*Note hh is effectively being multiplied by HI value 1*/

}

/*If heuristics table shows that a MED result is expected for C, when A is HI and B is HI,

then . . . */

if(TempTable[A_HI][B_HI]==MD) /*schema at A_HI,B_HI is MED*/

{

Cval=Cval+hh/2; /*Note hh is effectively being multiplied by MED value 0.5*/

}

/*If heuristics table shows that a LO result is expected for C, when A is HI and B is HI,

then . . . */

if(TempTable[A_HI][B_HI]==LO) /*schema at A_HI,B_HI is LO*/

{

Cval=Cval+hh*0; /*Note hh is effectively being multiplied by LO value 0*/

}

/*repeat this for all combinations of A and B, for example, hh, hm, hl, mh, mm, ml, lh, lm and ll*/

The resulting score from overlapping values of A and B on the matrix is a fuzzy value for C that can be defuzzified back into a crisp number. This predictive value of C is used to search the data set for actual values of C generated during the experiments. To qualify as an actual value of C in the illustrated embodiment, a data point must fit the predicted value of C in all or nearly all 17 experiments.

At S30, for each triplet, the agreement with the assertion in the rule table can be calculated based on average square of the residual, r², between the calculated C and the observed C for each experiment. Triplets having a low r² value fit the assertion better, and therefore are reported with higher confidence. During initial screening, only those triplets with r²<0.0015 were accepted, corresponding to an average error of 3% or less. This value is well below the error associated with the expression data. The value of r² was set a priori to minimize the amount of acceptable subsets, and may be varied according to the stringency requirements of a given screening process.

The following shows how fuzzified data may be applied to a decision matrix for a particular example, as shown in the pseudocode. The example shows A having a fuzzy value of 1.0 HIGH, 0 MED and 0 LOW and a B having a fuzzy value of 0.5 HIGH, 0.5 MED and 0 LOW.

A: 1.0 HI, 0 MED, 0 LO

B: 0.5 HI, 0.5MD, 0 LO

Filling out the matrix for all 9 possible values of A and B, i.e. hh, hm, hl, mh, mm, ml, lh, lm and ll, one can see that overlaps at fuzzA[HI]fuzzB[HI] (or hh) and fuzzA[HI]fuzzB[MED] (or hm) count=2

hh=1+0.5

hm=1+0.5

hl=0;

mh=0;

mm=0;

. . .

ll=0;

count=2, thus divide all by 3

hh=0.5

hm=0.5

hl=0;

. . .

11=0;

thus always has a sum of 1.

For the activator repressor schema, TempTable[A_HI][B_HI]=HI and

TempTable[A_HI][B_MD]=HI.

Thus Cval=0.5+0.5=1 /*Cval=hh+hm*/

Meaning that for this activator/repressor couple, we would expect C to be 1 (which means HI expression). If in truth, Cexperiment-0.98, then we calculate an rˆ 2 of (1-0.98)ˆ 2=0.0004

Occasionally, the data set of A and B failed to properly explore the decision matrix (i.e. A is almost always high, and B is almost always low), thus a second score called the variance was also assigned to the data set. The variance is defined as the statistical variance between the total hits in each box on the decision matrix. If the data set hits are evenly distributed throughout the decision matrix, then the variance score is low and the resulting predictions are credible. However, if the data set is poorly distributed, then the variance will be high and the predictions may or may not be believable due to the lack of combinations of A and B tested. The calculation of variance for the illustrated example, as referred to by S32 of FIG. 11, is set forth in the following pseudocode:

/*after each experiment, we have values of hh, hm, hl, mh, mm ml, lh, lm and ll which are put into a 3×3 table, called tablehold[2][2]. For the next experiment, new values of hh, hm, etc. are generated which are added to the current values of tablehold[2][2]. Once all experiments are run (17 in the case of the yeast data) then we use the 9 values in tablehold[2][2] to find how well distributed the values are. If all data is in HI,HI then we expect to get a poor distribution (i.e. tablehold[HI][HI] is very large and all other entries are low), and thus a high variance.*/

/*calculate mean values*/

mean=(tablehold[HI][HI]+tablehold[HI][MED]+.+tablehold[LO][LO])/9;

/*calculate variance*/

variance=((tablehold[HI][HI]−mean)*(tablehold[HI][HI]−mean)+(tablehold[HI][MED]−mean)*(tablehold[HI][MED]−mean)+(tablehold[HI][LO]−mean)*(tablehold[HI][LO]−mean)+

. . .

(tablehold[LO][LO]−mean)*(tablehold[LO][LO]−mean))/9;

As referred to by S34 of FIG. 11, a global view of how well the assertion fits the data may be gleaned from multiplying the r² value and the variance to give a general score. Thus, triplets with low r² values and low variance have the lowest score and also should be the most credible statements. Other data sets that are only low in one parameter may be filtered out because either the data set is biased or the fit is too poor. For the initial screen, only those A/B pairs with a variance of 1.5 or less were accepted.

Of the 6,321 known proteins in yeast, only 1898 (30%) genes were found whose expression levels were above the noise threshold and had a maximum value at least 3 times greater than the minimum value, ensuring that the observed signal change was statistically significant. Only these qualifying genes were processed by the fuzzy logic algorithm. Using an initial screening cutoff for r² and variance, the list of potential genes was narrowed down to the top 0.007% of all possible triplets, thereby forming the basis for initial calculations.

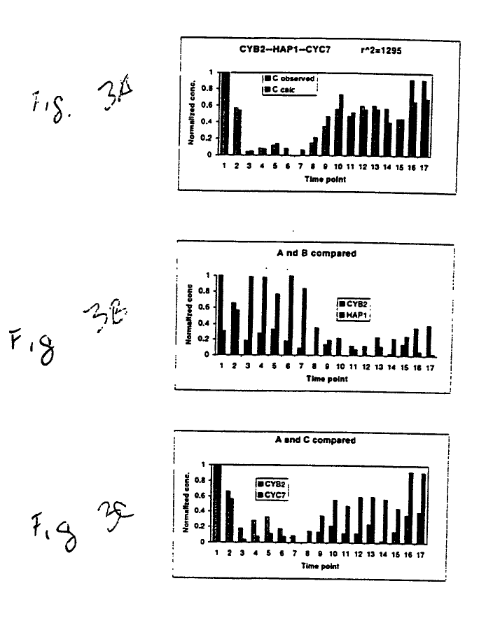

In order to evaluate and validate the algorithm, the best scoring triplets were examined to assess their biological relevance.. One of the best scoring triplets, CYB2-HAP1-CYC7, is shown in FIG. 3., wherein the top panel shows a correlation of the predicted and the observed values for CYC1 (C), while the middle panel shows the relationship of CYB2 (A) and HAP1 (B), and the bottom panel depicts the relationship of CYB2 (A) and CYC1 (C).

FIG. 3A shows the tight fit of the fuzzy logic prediction for CYC7 expression level as compared to the experimental data. The r² value for this triplet is 0.013 between the calculated expression data of CYC7 and the observed data (FIG. 3A), indicating a very high confidence for the correlation. Moreover, the expression data of CYB2 (A) and HAP (B) show a fairly wide range of values and are evenly distributed throughout the decision matrix (FIG. 3C). Thus, the variance score is low and the resulting predictions are credible. Of note is the fact that neither CYB2 (A) nor HAP1 (B) could be categorized in the same cluster as CYC7 (C) as shown in FIG. 3b. Thus, only by using the fuzzy logic algorithm of the illustrated embodiments of the invention could this triplet be uncovered.

Previous studies show that all three genes in this triplet are involved in yeast respiration process and the predicted relationships between the genes are borne out by experimental results in the literature (Fytlovich, 1993; Lodi, 1991). The transcription factor HAP1 has been shown to repress the nuclear encoding cytochrome gene CYC7 under anaerobic growth, but activate CYC7 under aerobic growth (Prezant, 1987). The fuzzy logic prediction suggests that HAP1 represses CYC7, which in turn accurately predicts that the cells used in this data set were primarily grown under anaerobic conditions.

The algorithm also predicts that CYB2 should activate CYC7, again in agreement with experimental findings. CYB2, L-(+)-lactate cytochrome c oxidoreductase is a soluble protein from the intermembrane of mitochondria. This protein transfers electrons from L-(+)-lactate to cytochrome c and is upstream of cytochrome c on the electron transport chain. Experimental findings indicate that CYB2 interacts preferentially with CYC7 during the electron transfer process (Fytlovich, 1993) and as such should positively regulate the expression of CYC7 as found by the algorithm.

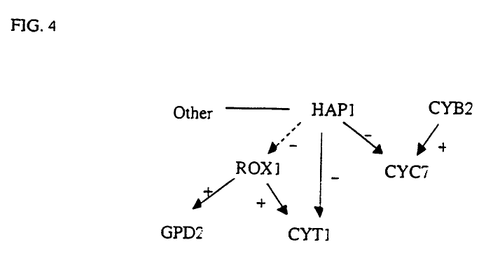

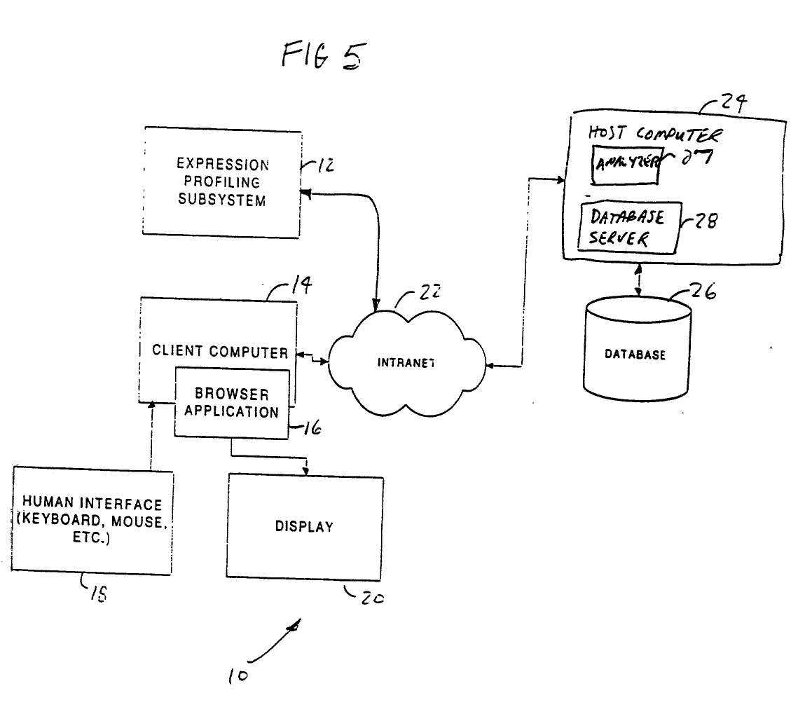

In further exploring the relationships revealed by this triplet, all triplets containing either HAP1 or HAP1 regulated genes were selected in an effort to generate an interconnected network describing the control roles of HAP1. The network predicted by the fuzzy logic algorithm is described in FIG. 4 and is highly consistent with the experimental data obtained from previous studies. Moreover, the fuzzy logic algorithm permitted functional identification of unidentified genes involved in this process, enabling hypothesis formation for future experimental tests. For example, previous studies show that an unidentified protein X masks the activation domain of HAP1 and allows HAP1 to act as a repressor under anaerobic conditions (Zhang, 1998; Hatch, 1999). Currently, protein X remains uncharacterized. However, fuzzy logic prediction suggests that several gene products, including YDL174C, YGL037C, YLR251W, YLR252W and YNL007C, could be this uncharacterized protein. Functionally, these proteins appear to repress HAP1's ability to activate CYC7, however further experiments are needed to determine the exact protein involved.

Both CYT1 and CYC1 are regulated by HAP1 (Schneider, 1991), however the fuzzy logic algorithm only uncovered a relationship for CYT1. On further study it was found that CYT1, like CYC7 discussed above, has a single HAP1 binding site, while the CYC1 promoter has two HAP1 binding sites (Prezant, 1987). A major consequence of the two cooperative sites of CYC1 is that the HAP1 modulation profile should be sigmoidal rather than linear. By contrast, the single promoter site in CYT1 and CYC7 should provide these genes a more linear reduction in activity as HAP1 expression changes. The fuzzy logic algorithm of the claimed invention is preferably limited to detecting linear relationships, although nonlinear proteins controlled by two redundant sites may be detected with less efficiency than linear relationships.

Experimentally, it has been shown that HAP1 regulates ROX1, a protein that encodes a repressor protein for hypoxic genes (Zitomer, 1997, Deckert, 1995). When cells are grown under aerobic conditions, heme accumulates to levels sufficient to induce ROX1 expression and the hypoxic genes are repressed. When cells are limited for oxygen, heme levels fall, ROX1 repressor levels are reduced and hypoxic gene expression is depressed. The relationship between HAP1 and ROX1 was not revealed by the fuzzy logic because expression of ROX1 is transient and highly unstable. Two genes, CYT1 and GPD2 were, however, found by the algorithm as the targets for the positive regulation by ROX1 while most of the hypoxic genes were not identified. This result suggests that the view of hypoxic gene regulation is correct in terms of the phenomenology but there is a great deal more complexity to ROX1 regulation.

Table 1 lists the most frequent occurring pairs of genes in triplets identified by the fuzzy logic algorithm, many of which appear to be biologically relevant. In several cases, both gene products function in the same cellular process. For example, AGP1 and MEP2 are often found together. Functionally AGP1 encodes a broad substrate range amino acid permease whose expression is subject to nitrogen repression, while MEP2 is a high-affinity ammonia permease induced by to nitrogen starvation. Thus, it makes sense that AGP1 and MED2 are found in the same triplet. Similarly, HAP1 is a transcription factor with a broad spectrum of targets including genes involved in sterol biosynthesis such as FAA1 and ARE2. FAA1 is long chain fatty acyl:CoA aynthetase in lipid metabolism and protein N-myristolation. ARE2 is sterol-ester synthetase in ergosterol esterification. In general, HAP1 is known as a repressor, thus the fact that this algorithm identifies HAP1 as repressing FAA1 and ARE2 is consistent with known biological data.

YGP1 and CBF2 represent a very interesting relationship predicted by the algorithm. YGP1 is a highly glycosylated secreted protein involved in cellular adaptations prior to stationary phase. The gene is expressed at a basal level during logarithmic growth and induced up to 50-fold above basal level when cells enter stationary phase. Conversely, CBF2 is a chromosome centromere binding protein in the multisubunit protein complex and involved in cellular replication and cell division. CBF2 expression is expected to be high during logarithmic growth, but low during stationary phase. This reciprocal relationship between YGP1 and CBF2 is well represented in the predicted results as the two proteins were found in either B or C positions depending upon the protein as the activator in the triplet. When CDC46, a protein enriched in non-dividing cells, is the activator, YGP1 is found as the target in the triplet. When proteins promoting cell cycle progression such as CDC45, RPL14A, ZDS2, NHP6A and IPL1 are activators, CBF2 acts as the target.

In addition, pairs of genes are also predicted by the fuzzy logic algorithm wherein one or both of the genes are uncharacterized. By analogy to the examples shown above, it may be possible to infer the cellular function of these unknown proteins by examining what known proteins are found to associate with the set. This ability to bootstrap functional information out of the expression data should be particularly useful in analyzing human data, where a much larger percentage of proteins are uncharacterized.

Since the fuzzy logic algorithm of the illustrated embodiment was used to search for activator-repressor-target triplets, a disproportionately large number of triplets were expected to include transcription factors. After an initial screening, the results revealed that transcription factors were found 30% more frequently than would be expected from a random distribution. When only looking at the 100 best scoring triplets, transcription factors were found 90% more frequently than at random.

In general, the findings of the algorithm agree well with experimental results from the literature. Such findings are rational, given that the algorithm searches for relationships that fit logical scientific understanding of how an activator, repressor, and target should interact. The fuzzy logic algorithm embodiments illustrated herein only searched for triplets of activator, repressor, and target genes. The choice of the activator-repressor model was based on simplicity in order to demonstrate that this technology is capable of yielding biologically meaningful results. The technique, however, is not limited to triplet subsets, and can be applied to different size subsets of data, corresponding to other relationships and more complicated systems. Examples include, inter alia, other classes of relationships such as co-activators and co-repressors, or more complicated systems that involve genes whose transcription is regulated by any number of transcription factors. It is contemplated that the technology may be useful in describing complete general networks of gene interactions.

Using fuzzy logic to analyze expression data does have some limitations. To a first approximation, the interaction between multiple proteins is essentially linear, thus the algorithm searches for linear behavior. However, in the event of multiple redundant promoter binding sites (such as the two HAP1 binding sites on the CYC1 gene), this linear approximation is not accurate, causing the algorithm to overlook these biologically relevant connections. Such a situation could be remedied by including a more sophisticated “fuzzification” step to include nonlinear effects. While useful, this added complexity may only correct for a few missed connections while edging out many of the more common near linear relationships. Still, gross nonlinear relationships between biological moieties may be assessed using fuzzy logic.

In practice, the fuzzy logic algorithm found a disproportionately large number of transcription factors in the roles of activators and repressors, although not all of the activators and repressors found were transcription factors. Two possible reasons for this discrepancy are 1) transcription factors are expressed at low levels and thus are difficult to detect; and 2) enzymes can indirectly regulate transcription. Transcription factors are generally present only at a very low concentration, thus changes in transcription factor expression levels can be difficult to detect using current expression profiling techniques. Presumably, if expression profiling technology were to become more sensitive, the fuzzy logic algorithm would detect an even greater bias of transcription factors in the activator and repressor roles. However, in many cases the expression level of a particular protein is not governed by the expression of a transcription factor, but instead by the concentration of some intracellular compound, such as Ca2+ concentration or cAMP levels, which in turn are controlled by enzymes inside the cell. In these cases, changes in the expression level of the enzyme have a “transcription-factor-like” effect and would be detected by the algorithm as an activator or repressor. From an drug design point of view, these “transcription-factor-like” enzymes are possibly more interesting than true transcription factors, because it is generally easier to change the activity of an enzyme in the cytosol with a drug than to block a true transcription factor in the nucleus.

Although the validation of this algorithm was performed using GeneChip® data, the fuzzy logic algorithm should work equally well with other expression profiling techniques such as Sequential Analysis of Gene Expression (SAGE). SAGE has the advantage that it can detect completely unknown proteins, while GeneChip® technologies require that at least the sequence of a protein's mRNA be known. This ability to detect unknown proteins would be particularly well suited to the functional characterization that the fuzzy logic algorithm makes possible.

An additional advantage to the fuzzy logic algorithm is that data can come from any source within an organism (tissue, cell type, treatment, or physiological state) and the output actually will be improved by deeper and more diverse data set. The reason for this improvement is that the algorithm needs to observe changes in the expression level of a protein relative to changes in other expression levels. Each new data set provides a different set of expression levels that can be tested to see if they fit the proposed regulatory model. In our studies, many data sets were eliminated solely because they did not sufficiently explore the combinations of expression levels (too high a sigma value), making their predictions impossible to believe. By including data sets from cells in different states, the algorithm gains more information about the details of the regulatory network.

One application of this algorithm is to independently validate or discover drug targets. Traditional techniques for drug target discovery require a detailed understanding of the biology underlying the disease, which can be slow and difficult to obtain. In contrast, expression profiling is a rapid high-throughput process, providing a large amount of information about the cell in a form that could be easily processed on a computer. By using a fuzzy logic approach to analyzing expression profile data, it is possible to confirm the mechanism of a known target. Moreover, because the fuzzy logic algorithm does not require biological information about the gene, genes encoding proteins with unknown functions can be included just as easily as genes encoding proteins with known functions. This ability to identify functional clues for uncharacterized genes is a great advantage in drug target discovery because potential drug targets then can be followed up with the detailed biology.

The invention may be implemented by hardware or a combination of hardware and software. The software may be recorded on a medium for reading into a computer memory and executing. The medium may be, but is not limited to, for example, one or more of a floppy disk, a CD ROM, a writable CD, a Read-Only-Memory (ROM), and an Electrically Alterable Programmable Read Only Memory (EAPROM).

While the invention has been described by way of example embodiments, it is understood that the words which have been used herein are words of description, rather than words of limitation. Changes may be made, within the purview of the appended claims, without departing from the scope and spirit of the invention in its broader aspects. Although the invention has been described herein with reference to particular means, materials, and embodiments, it is understood that the invention is not limited to the particulars disclosed. The invention extends to all equivalent structures, means, and uses which are within the scope of the appended claims.

REFERENCES

1. Cho, R. J., M. J. Campbell, E. A. Winzeler, L. Steinmetz, A. Conway, L. Wodicka, T. G. Wolfsberg, A. E. Gabrielian, D. Landsman, D. J. Lockhart, and R. W. Davis. A Genome-Wide Transcriptional Analysis of the Mitotic Cell Cycle. Mol. Cell 2:65-73, 1998.

2. Cox, E. Fuzzy Fundamentals. IEEE Spectrum, 58-61, 1992

3. Deckert, J., R. Perini, B. Balasubramanian, and R. S. Zitomer. Multiple Elements and Auto-Repression Regulate Rox1, a Repressor of Hypoxic Genes in Saccharomyces cerevisiae. Genetics Soc. Of America 139:1149-1158, 1995.

4. Fytlovich, S., M. Gervais, C. Agrimonti, and B. Guiard. Evidence for an interaction between the CYP1 (HAP1) activator and a cellular factor during heme-dependent transcriptional regulation in the yeast Saccharomyces cerevisiae. EMBO J. 12:1209-1218, 1993.

5. Hach, A., T. Hon, L. Zhang. A New Class of Repression Modules Is Critical for Heme Regulation of the Yeast Transcriptional Activator Hap1. Mol. Cell. Biol. 19:4324-4333, 1999

6. Lodi, T., and B. Guiard. Complex Transcriptional Regulation of the Saccharomyces cerevisiae CYB2 Gene Encoding Cytochrome b₂: CYP1(HAP1) Activator Binds to the CYB2 Upstream Activation Site UAS1-B2. Mol. Cell. Biol. 11:3762-3772, 1991.

7. Prezant, T., K. Pfeifer, and L. Guarente. Organization of the Regulatory Region of the Yeast CYC7 Gene: Multiple Factors Are Involved in Regulation. Mol. Cell. Biol. 7:3252-3259, 1987.

8. Schneider, J. C., and L. Guarente. Regulation of the Yeast CYT1 Gene Encoding Cytochrome c₁ by HAP1 and HAP2/3/4. Mol. Cell. Biol. 11:4934-4942, 1991.

9. Tavazoie, S., J. D. Hughes, M. J. Campbell, R. J. Cho, and G. M. Church. Systematic Determination of Genetic Network Architecture. Nature Genetics 22:281-285, 1999.

10 Yakhini, Z., and Ben-Dor, A. Clustering Gene Expression Patterns. Technical Report HPL-98-190, HP-labs Israel, 1998.

11. Zadeh, L. A. Fuzzy Logic and its Application to Approximate Reasoning. Information Processing, 74:591-594, 1974.

12. Zhang, L., A. Hach, and C. Wang. Molecular Mechanism Governing Heme Signaling in Yeast: a Higher-Order Complex Mediates Heme Regulation of the Transcriptional Activator HAP1. Mol. Cell. Biol. 18:3819-3828, 1998.

13. Zitomer, R. S., M. P. Limbach, A. M. Rodriguez-Torres, B. Balasubramanian, J. Deckert, and P. M. Snow. Approaches to the Study of Rox1 Repression of the Hypoxic Genes in the Yeast Saccharomyces cerevisiae. Methods in Enzymology 11:279-288, 1997.

TABLE 1
Frequent pairs of genes identified by the fuzzy logic algorithm

| Name | Position | Description | # repeats |
|---|---|---|---|
| AGP1 | A | Broad substrate range permease which | |
| | | transports asparagine | |
| | | and glutamine | |
| MEP2 | C | Ammonia transport protein | 79 |
| YGP1 | B or C | involved in cellular adaptations prior | |
| | | to stationary phase | |
| CBF2 | B or C | component (Cbf3a) of the multisubunit | 71 |
| | | ‘Cbf3’ kinetochore | |
| | | protein complex | |
| PEP5 | C | peripheral vaculor membrane protein; | |
| | | putative Zn-finger | |
| | | protein | |
| MEP2 | A | Ammonia transport protein | 66 |
| PEP5 | C | peripheral vaculor membrane protein; | |
| | | putative Zn-finger | |
| | | protein | |
| ARG4 | A | argininosuccinate lyase | 63 |
| CPS1 | B | carboxypeptidase yscS | |
| HES1 | C | Protein implicated in ergosterol | 57 |
| | | biosynthesis | |
| GAP1 | B | general amino acid permease | |
| SPO13 | C | a negative regulator of M-phase during | 52 |
| | | melosis | |
| HAP1 | B | positive regulator of cytochrome C | |
| | | genes CYC1 and CYC7 | |
| FAA1 | C | long chain fatty acyl:CoA synthetase | 44 |
| HAP1 | B | positive regulator of cytochrome C | |
| | | genes CYC1 and CYC7 | |
| ARE2 | C | Acyl-CoA cholesterol acyltransferase | |
| | | (sterol-ester synthetase) | 39 |
| HAP1 | B | positive regulator of cytochrome C | |
| | | genes CYC1 and CYC7 | |
| GAP1 | C | general amino acid permease | 38 |

What is claimed is:

1. A method for managing and analyzing information obtained from differential expression of genetic information in biological cells, the method comprising:
receiving crisp input data from sets of expression data from control and treatment sets of cell-derived samples representing a direction and a magnitude of regulation of each one of a higher number of different genes or proteins;
fuzzifying the crisp input data to provide fuzzified values;
applying a set of heuristic rules to the fuzzified values to generate a predicted value of a data point C;
defuzzifying the predicted value of C; and
determining a confidence level of the predicted value of C.

2. The method of claim 1, further comprising:
filtering the crisp data to ensure the crisp data are above a predetermined noise level.

3. The method of claim 2, wherein the applying of the set of heuristic rules is performed by using a decision matrix.

4. The method of claim 2, wherein the determining of the confidence level of the predicted value of C comprises:
calculating a difference r between the defuzzified predicted value and an observed value of C; and
squaring r to provide r² and comparing r² to a predetermined value, a value of r² being smaller than the predetermined value indicating a high confidence level in the defuzzified predicted value of C.

5. The method of claim 4, wherein the determining of the confidence level of the predicted value of C further comprises:
determining a distribution variance of the fuzzified values; and
obtaining a general score, for predicting credibility of decision matrix predictions, based upon the distribution variance and the value of r².

6. The method of claim 2, wherein the filtering comprises:
accepting the crisp input data only when one of the crisp input data having a value greater than all other ones of the crisp input data is at least three times larger than another one of the crisp input data having a value less than all other ones of the crisp input data.

7. The method of claim 5, further comprising:
accepting only ones of the input data satisfying r²<.0015.

8. The method of claim 5, wherein the obtaining of the general score comprises multiplying the distribution variance and the value of r².

9. A system for managing and analyzing information obtained from differential expression of genetic information in biological cells, the system comprising:
a data receiver for receiving crisp input data from sets of expression data from control and treatment sets of cell-derived samples;
a fuzzifier for fuzzifying the crisp input data to provide fuzzified values;
a heuristic rules applier for applying a set of heuristic rules to the fuzzified values to generate a predicted value of a data point C;
a defuzzifier for defuzzifying the predicted value of C; and
a confidence level determiner for determining a confidence level of the predicted value of C.

10. The system of claim 9, further comprising:
a filter for filtering the crisp input data to ensure the crisp data are above a predetermined noise level.

11. The system of claim 10, wherein the heuristic rules applier uses a decision matrix.

12. The system of claim 10, wherein the confidence level determiner comprises:
a calculator for calculating a difference r between the defuzzified predicted value and an observed value of C; and
a squarer for squaring r to provide r² and for comparing r² to a predetermined value, a value of r² being smaller than the predetermined value indicating a high confidence level in the defuzzified predicted value of C.

13. The system of claim 12, wherein the confidence level determiner further comprises:
a variance determiner for determining a distribution variance of the fuzzified values; and
a scorer for obtaining a general score, for predicting credibility of decision matrix predictions, based upon the distribution variance and the value of r².

14. The system of claim 10, wherein the filter comprises:
an accepter for accepting the crisp input data only when one of the crisp input data having a value greater than all other ones of the crisp input data is at least three times larger than another one of the crisp input data having a value less than all other ones of the crisp input data.

15. The system of claim 13, wherein the scorer comprises a multiplier for multiplying the distribution variance and the value of r².

16. A machine-readable medium having recorded thereon machine-readable information, such that when the machine-readable information is read and executed by a computer, the machine-readable information causes the computer to:
receive crisp input data from sets of expression data from control and treatment sets of cell-derived samples representing a direction and a magnitude of regulation of each one of a higher number of different genes or proteins;
fuzzify the crisp input data to provide fuzzified values;
apply a set of heuristic rules to the fuzzified values to generate a predicted value of a data point C;
defuzzify the predicted value of C; and
determine a confidence level of the predicted value of C.

17. The medium of claim 16, wherein the machine-readable information further causes the computer to:
filter the crisp data to ensure the crisp data are above a predetermined noise level.

18. The medium of claim 17, wherein the computer applies the set of heuristic rules by using a decision matrix.

19. The medium of claim 17, wherein the machine-readable information causes the computer to determine the confidence level of the predicted value of C by:
calculating a difference r between the defuzzified predicted value and an observed value of C; and
squaring r to provide r² and comparing r² to a predetermined value, a value of r² being smaller than the predetermined value indicating a high confidence level in the defuzzified predicted value of C.

20. The medium of claim 19, wherein the machine-readable information further causes the computer to determine the confidence level of the predicted value of C by:
determining a distribution variance of the fuzzified values; and
obtaining a general score, for predicting credibility of decision matrix predictions, based upon the distribution variance and the value of r².

21. The medium of claim 17, wherein the machine-readable information causes the computer to filter the crisp input data by:
accepting the crisp input data only when one of the crisp input data having a value greater than all other ones of the crisp input data is at least three times larger than another one of the crisp input data having a value less than all other ones of the crisp input data.

22. The medium of claim 20, wherein machine-readable information causes the computer to obtain the general score by multiplying the distribution variance and the value of r².
